(19) United States
(12) Patent Application Publication
(10) Pub. No.: US 2025/0248902 A1
(43) Pub. Date: Aug. 7, 2025

(54) Curable Composition for Dental Purposes

(71) Applicant: VITA ZAHNFABRIK H. RAUTER GMBH & CO. KG, Bad Säckingen (DE)

(72) Inventors: Thorsten PALMER, Tamstedt (DE); Christoph PFEIFER, Bad Säckingen (DE)

(21) Appl. No.: 18/853,330

(22) PCT Filed: Apr. 11, 2023
(86) PCT No.: PCT/EP2023/059434

§ 371 Date: Oct. 1, 2024

(30) Foreign Application Priority Data
Apr. 13, 2022 (EP) 22168198.4

(51) Int. Cl. A61K 6/887; A61K 6/76; A61K 6/79; C08L 33/10

(52) U.S. Cl. CPC A61K 6/887; A61K 6/76; A61K 6/79; C08L 33/10; C08L 2203/02; C08L 2205/025; C08L 2205/035; C08L 2207/53

(57) ABSTRACT

The present invention relates to a curable composition for dental purposes. The invention is characterized in that the composition comprises core-shell impact modifiers and 0.1 to 10 wt. % of a binder based on long-chain mono-functional (meth)acrylic acid esters, wherein the ester group has a carbon chain with at least 3 carbon atoms. The invention also relates to the use of the composition as a dental material or a dental auxiliary material, to a dental product made of the composition, and to a method for producing the dental product.

The present invention relates to a curable composition for dental purposes, which is characterized in that it includes core-shell impact modifiers and from 0.1 to 10% by weight of a binder based on longer-chain monofunctional (meth)acrylates, in which the ester group has a carbon chain with at least 3 carbon atoms, the use of the composition as a dental material or dental auxiliary, a dental product prepared from the composition, and a process for preparing said dental product.

Curable compositions as employed, for example, in additive manufacturing are known to those skilled in the art, and have also been introduced into the dental field, so that it has been possible to prepare dental products, such as dental restorations, with high precision and individually matched in the meantime.

EP 3 564 282 describes curable compositions for use in a high temperature lithography-based photopolymerization process, including a specific oligomeric dimethyl acrylate as a glass transition modifier.

US 2021/0017302 discloses a resin composition comprising: one or more photoinitiators; one or more hard cross-linkers, wherein the one or more hard cross-linkers have at least two reactive groups and are present at 20-75 wt % (based on the total weight of the composition); and one or more soft cross-linkers and/or one or more reactive diluents, wherein the one or more soft cross-linkers have at least two reactive groups and are present at 2-70 wt % (based on the total weight of the composition) and/or the one or more reactive diluents have only one reactive group and are present at 2-70 wt % (based on the total weight of the composition).

WO 2014/078537 relates to a composition for making three-dimensional dental products comprising a mixture of at least 1% methyl methacrylate and/or ethyl methacrylate, 5 to 10% of silicone-acrylic-based rubber impact modifiers, the core being silicone, and the shell being PMMA acrylic, which does not dissolve but swells and forms a colloid at room temperature or elevated temperature, 1 to 90% of at least one mono- or multifunctional (meth)acrylate, 0 to 60% of at least one inorganic filler, 0 to 60% of at least one organic filler, 0 to 10% of pigments, and 0.01 to 10% of photoinitiators.

WO 2020/182810 relates to a polymerizable radiation-curable composition comprising (i) monomers and (ii) at least one component, in which the monomers comprise at least (a) one first monomer with a homopolymer TG (glass transition temperature) which is greater than or equal to 120° C. and at least (b) one second monomer with a homopolymer TG (glass transition temperature) which is less than or equal to 100° C., wherein at least one of the at least one first monomer or the at least one second monomer has an alicyclic group, and said at least one further component comprises at least one photoinitiator for the UV and/or the Vis range or a photoinitiator system for the UV and/or Vis range.

EP 3 925 994 describes a photo-curable composition including a di-(meth)acrylic monomer (A) having two (meth)acryloyloxy groups and two urethane bonds, an acrylic monomer (B) having one acryloyl group, and a photopolymerization initiator.

WO 2019/204807 discloses a thermoset composition suitable for use in additive fabrication processes including high concentrations of urethane methacrylate oligomers having at least one polymerizable group, and a number average molecular weight from 750 to 4000 g/mol, an effective quantity of a monofunctional reactive diluent monomer, a defined concentration of a network building agent having a molecular weight that is lower than that of the urethane methacrylate oligomer, optionally a photoinitiator, and optionally, additives.

EP 3 494 954 relates to a photocurable composition, which is used for manufacturing a dental prosthesis or the like by stereolithography, including: a photopolymerization initiator; and a (meth)acrylic monomer component including an acrylic monomer (X) having no aromatic rings and having a ring structure other than an aromatic ring and two or more acryloyloxy groups in one molecule and having an Mw of from 200 to 800, and at least one of a (meth)acrylic monomer (A) having one or more ether bonds and two (meth)acryloyloxy groups in one molecule and having an Mw of from 200 to 400, a (meth)acrylic monomer (B) having a ring structure other than an aromatic ring and one (meth)acryloyloxy group in one molecule and having an Mw of from 130 to 240, a (meth)acrylic monomer (C) having a hydrocarbon skeleton and two (meth)acryloyloxy groups in one molecule and having an Mw of from 190 to 280, and a (meth)acrylic monomer (D) having one or more aromatic rings and one (meth)acryloyloxy group in one molecule and having an Mw of from 140 to 350.

US 2018/0000570 describes a method for forming a dental component, in which a polymerizable composition is used that contains core-shell impact modifiers, methyl methacrylate, and an initiator.

US 2009/0192240 discloses hardenable dental composite materials, comprising i) non-agglomerated nanofillers having particle sizes of about 1 to about 50 nm; and ii) core-shell polymer particles with an elastic core compound, and a substantially non-elastic shell compound.

Compositions that are conventionally employed in additive manufacturing have the disadvantage that, because of their low impact strength, they have a breaking behavior that makes them unsuitable for dental applications. The impact strength necessary for dental applications is achieved by adding impact modifiers, or the use of phase-separated plastic materials. In order to achieve sufficient incorporation of the impact modifiers into the polymer matrix, components with a high vapor pressure are usually employed, which are highly volatile and cause the dental product to possess an unpleasant odor. In addition, the addition of the impact modifiers adversely affects the mechanical properties of the composition and has an impact, in particular, on fracture toughness and fracture work, but also on bending strength. Therefore, there is still a need for improved compositions for the preparation of dental products.

Surprisingly, it has been found that the pollutant input to the environment from the processing of such materials into dental products, which is mostly characterized by an unpleasant smell, can be reduced by employing compounds having a lower vapor pressure, without resulting in a negative impact on printability of such compositions, for example, within the scope of additive manufacturing methods.

Therefore, the present invention firstly relates to a curable composition for dental purposes, comprising, respectively based on the total weight of the composition:
a) from 0.1 to 10% by weight of a binder based on a monomeric monofunctional (meth)acrylate, in which the ester group has a carbon chain of 3 or more carbon atoms;
b) from 1 to 15% by weight of one or more core-shell impact modifiers; and
c) from 1 to 90% by weight of a matrix material; and
d) from 0.01 to 3% by weight of at least one initiator.

Surprisingly, it has been found that the composition according to the invention can not only usually reduce the unpleasant odor of dental restorations, but in addition, the composition has also a higher temperature stability in the liquid state, and thus a better processability can be achieved. In particular, the use of a longer-chain monofunctional (meth)acrylate as a binder provides the composition with mechanical properties that allow it to be used in the dental field. Thus, it has surprisingly been found that the addition of the binder according to the invention could improve the fracture toughness and fracture work of the dental composition while the bending strength remained constant.

The ester group of the binder includes a carbon chain of 3 or more carbon atoms, in which said chain may have further substituents. In a preferred embodiment, the carbon chain includes 4 or more carbon atoms, preferably 5 or more carbon atoms. Preferably, however, the carbon chain has not more than 16 carbon atoms, in order not to adversely affect the processability of the composition. In a particularly preferred embodiment, the binder is an aliphatic monofunctional (meth)acrylate with an ester group having a carbon chain of C₃H₇ to C₁₆H₃₃, preferably C₃H₇ to C₆H₁₃. In a particularly preferred embodiment, the binder is selected from the group consisting of propyl (meth)acrylate, butyl (meth)acrylate, pentyl (meth)acrylate, and hexyl (meth)acrylate, and their isomers. Mixtures of the mentioned compounds may also be used.

In a preferred embodiment, the content of binder in the composition according to the invention is from 0.2 to 7% by weight, preferably from 0.5 to 5% by weight, based on the total weight of the composition.

In order to avoid the unpleasant odors associated with short-chain (meth)acrylates, especially methyl (meth)acrylate and ethyl (meth)acrylate, the composition according to the invention is preferably free of such compounds. In a preferred embodiment, the content of monomeric methyl (meth)acrylate and/or ethyl (meth)acrylate in the composition according to the invention is preferably not more than 0.5% by weight, more preferably less than 0.5% by weight, especially less than 0.1% by weight, respectively based on the total weight of the composition.

The impact modifiers contained in the composition according to the invention are preferably selected from the group of core-shell particles based on silicone/(meth)acrylates and/or (meth)acrylates/(meth)acrylates. More preferably, dais impact modifiers are core-shell particles with a core of silicone and a shell of PMMA, or core-shell particles with a core based on (meth)acrylates and a shell of PMMA. Within the scope of the present invention, it has been found that such impact modifiers, especially in compositions employed in additive manufacturing, contribute to the fact that products having a high mechanical strength and excellent physical properties are obtained.

Further, the composition according to the invention contains at least one matrix material. Preferably, said matrix material is a mono- or multifunctional (meth)acrylate or mixtures of this compound. More preferably, said matrix material is different from said binder material.

Compounds known to the person skilled in the art of dentistry may be used as the matrix material. Preferably, said matrix material is polymerizable (meth)acrylate compounds, such as cyclohexyl (meth)acrylate, 4-tert-butylcyclohexyl (meth)acrylate, tetrahydrofurfuryl (meth)acrylate, 2-phenoxyethyl (meth)acrylate, allyl acrylate, isobornyl (meth)acrylate, stearyl (meth)acrylate, phenoxybenzyl (meth)acrylate, o-phenylphenolethyl (meth)acrylate, 1,14-tetradecanyl dimethacryate, dimethyloltricyclodecanyl diacrylate, glyceryl diacrylate, glyceryl triacrylate, ethylene glycol diacrylate, diethylene glycol diacrylate, triethylene glycol dimethacrylate, tetraethylene glycol di(meth)acrylate, 1,3-propanediol diacrylate, 1,3-propanediol dimethacrylate, trimethylolpropane tri (meth)acrylate, 1,2,4-butanetrio trimethacrylate, 1,4-cyclohexanediol diacrylate, 1,4-cyclohexanediol dimethacrylate, 1,6-hexanediol di(meth)acrylate, pentaerythritol triacrylate, pentaerythritol tetraacrylate, pentaerythritol tetramethacrylate, sorbitol hexacrylate, 2,2-bis[4-{2-hydroxy-3-acryloyloxypropoxy)phenyl]propane and 2,2-bis[4-(2-hydroxy-3-methacryloyioxypropoxy)phenyl]propane (Bis-GMA). In particular, the matrix material is selected from the group consisting of tricyclodecanedimethanol dimethacrylate, ethylene glycol phenyl ether methacrylate, isobornyl methacrylate, 3,3,5-trimethylcyclohexyl methacrylate, and tricyclodecanyl acrylate, and mixtures thereof.

In a preferred embodiment, the composition according to the invention includes up to 70% by weight of at least one difunctional urethane (meth)acrylate, based on the total weight of the composition. In a further preferred embodiment, the content of difunctional urethane (meth)acrylate is at least 25% by weight, more preferably 30 to 45% by weight, respectively based on the total weight of the composition. Aliphatic polyesters/polyethers based on urethane di(meth)acrylate oligomers, especially UDA and UDMA, are preferably used as said difunctional urethane (meth)acrylate. In this way, in particular, the elongation at break of products prepared from the composition according to the invention could be increased.

The curable composition according to the invention further contains initiators that initiate the curing of the composition. Such initiators may be thermal initiators or photoinitiators. Preferably, the initiator is contained in the curable composition in an amount of from 0.01 to 2% by weight, especially from 0.5 to 1.5% by weight, based on the total weight of the curable composition. In a preferred embodiment, the initiator is selected from the group consisting of benzophenones, benzoins or α-diketones, in order to make the composition photocurable. Preferred initiators further include camphorquinone (CQ), or cationic initiators, such as 4-octyloxyphenyl phenyliodonium hexafluoroantimonate (OPPI). Further suitable initiators are those selected from the class of acylphosphine oxides, such as nonoacylphosphine oxides, bisacylphosphine oxides and triacylphosphine oxide, especially 2,4,6-trimethylbenzoyl diphenyl phosphinate (TPO), and ethyl (2,4,6-trimethylbenzoyl)phenylphosphinate (TPO-L). The curing of the composition according to the invention is preferably initiated by irradiation with light, preferably having a wavelength of from 365 to 500 nm, more preferably from 385 to 405 nm, so that instruments that are common in the dental field can be employed. Within the scope of the present invention, it is also preferred to use mixtures of thermal initiators and photoinitiators. In this way, the composition according to the invention can be shaped, for example, in a 3D printer, in which light-based systems, such as DLP, LCD or stereolithographs, are preferred. The final curing can then be effected by another irradiation or thermal treatment.

The curable composition according to the present invention may include further components in order to adapt its properties according to the intended application. Therefore, an embodiment is preferred in which said curable composition further includes inorganic fillers. Such fillers may be contained in an amount of up to 60% by weight in the composition according to the invention, based on the total weight of the composition. Preferably, the inorganic fillers are selected from the group consisting of silica, zirconia, aluminum oxide, alumina, apatite, titanium dioxide, and glass microbeads. Such inorganic fillers have proven particularly advantageous if the curable composition is used for the manufacturing of artificial teeth.

In a further preferred embodiment, the curable composition of the present invention further contains organic fillers, which may be present in an amount of up to 60% by weight, based on the total weight of the compositions. Such organic fillers are preferably those selected from the group consisting of acrylate beads, especially PMMA beads, and rubber beads. Such fillers may be employed, in particular, in order to improve the shrinking of the composition during the curing and its mechanical properties.

Especially in the cases where the composition according to the invention is used for preparing artificial teeth, the composition may further contain coloring substances. The amount of coloring substance in the composition depends on the desired color result, but in a preferred embodiment, it is not more than 10% by weight, more preferably less than 2.5% by weight, especially from 0.1 to 2% by weight, based on the total weight of the composition. Both organic and inorganic substances may be employed as coloring substances, wherein inorganic color pigments are preferred, in particular, since the best results can be achieved using them as far as the optical properties of dental restorations are concerned.

The curable composition according to the present invention preferably has a bending modulus of less than 5 GPa, preferably less than 5 GPa, especially from 2 to 4 GPa, in the cured state, as determined according to DIN EN ISO 20795-1 (2013) on printed specimens.

Said curable composition is preferably a one-part composition. Therefore, tedious mixing, as required for two-part compositions, can be omitted.

In particular, the curable composition is to be employed for dental purposes. Therefore, an embodiment is preferred in which the composition is printable and can be processed in additive manufacturing processes. In this way, for example, dental restorations, such as artificial teeth, having a high color and matching precision can be provided. Further, an embodiment is preferred in which the curable composition in a cured form is machinable, in order that manual corrections can be made, for example.

In particular, the curable composition of the present invention is tuned to the use for dental purposes. Therefore, the present invention further relates to the use of a curable composition according to the present invention as a dental material. Thus, the composition according to the invention is used, in particular, for the preparation of dental restorations, such as artificial teeth and full and partial prostheses, and other dental products, such as correction measures of the tooth position, or as an auxiliary for dental applications, for example, for the preparation of models and templates.

The present invention further relates to a dental product prepared from a curable composition according to the present invention. In particular, such a dental product is dental restorations, such as artificial teeth and full and partial prostheses, correction measures of the tooth position, such as braces or splints. In another preferred embodiment, the composition according to the invention is used as an auxiliary for dental applications, for example, for the preparation of models or templates.

The present invention further relates to a process for preparing a dental product by introducing a curable composition of the present invention into a device for additive manufacturing, and cured. Preferably, the curable composition is delivered layer by layer, and the individual layers are interconnected, for example, by irradiating with light. The delivery of the composition can be done, for example, on the basis of previously acquired data from the patient, in order to achieve a perfect fit. The final curing of the product is preferably effected by another irradiation or thermal treatment.

The present invention is further illustrated by means of the following Examples, which should by no means be understood as limiting the idea of the invention.

### EXAMPLES

For the preparation of the compositions, the following materials were employed, wherein the values in the Table are to be understood as weight percentages each.
UDA/UDMA: urethane diacrylate/urethane dimethacrylate
MA: Matrix material, mixture of mono- and multifunctional (meth)acrylates, obtainable from Arkema, France, Sartomer family of products
MMA: Methyl methacrylate
EMA: Ethyl methacrylate
Binder 1: Butyl 2-methyl-2-propenoate
Binder 2: Butyl 2-methylprop-2-enoate
Binder 3: Isopropyl methacrylate
Impact modifier 1: Core-shell (silicone/PMMA)
Impact modifier 2: Core-shell ((meth)acrylate/PMMA)
Initiator: TPO-L

| Example | A | B | C | D* | E | F | G* |
|---|---|---|---|---|---|---|---|
| UDA/UDMA | 30 | 35 | 30 | 35 | 23.4 | 23.4 | 23.4 |
| MA | 60.4 | 55.5 | 58 | 56 | 56.1 | 56.1 | 56.2 |
| MMA | 0.8 | — | — | — | — | — | — |
| EMA | 0.8 | — | — | — | — | — | — |
| Binder 1 | — | — | 4 | — | 3.3 | — | — |
| Binder 2 | — | 1 | — | — | — | — | — |
| Binder 3 | | | | | | 3.3 | |
| Impact modifier 1 | 4 | 7.5 | 4 | 7.5 | 6.3 | 6.3 | 6.3 |
| Impact modifier 2 | 3 | — | 3 | — | — | — | — |
| Silica | — | — | — | — | 10.0 | 10.0 | 10.0 |
| Initiator | 1 | 1 | 1 | 1.5 | 0.9 | 0.9 | 0.9 |
| Bending strength [MPa] | 60.8 | 75.4 | 55.4 | 30.4 | 85.2 | 86.7 | 84.6 |
| Bending modulus [MPa] | 1757 | 2336 | 1601 | 782 | 2374 | 2388 | 2297 |
| Fracture work [J/m²] | 1017 | 1441 | 853.2 | 115 | 878 | 704 | 678 |
| kₘₐₓ [MPa m^{1/2}] | 1.75 | 2.1 | 1.7 | 0.94 | 2.71 | 2.46 | 2.2 |

*Comparative compositions

The mechanical properties were determined as follows:

Bending strength and bending modulus: according to DIN EN ISO 20795-1 (2013) on printed specimens

Fracture work and fracture toughness (kₘₐₓ): by analogy with DIN EN ISO 20795-1 (2013), wherein the specimens were printed including an initial crack and notch, and the measurement was made outside the water bath.

As can be seen from the data of the Table, the comparative compositions D and G, which do not contain a binder, show inferior mechanical properties, which can be improved by adding compounds serving as binders (cf. compositions A-C, E and F). As can be further seen from the Table, The replacement of the binders MMA and EMA, which are to be considered critical, by longer-chain acrylates in compositions B and C does not adversely affect the mechanical properties. Rather, the fracture work and fracture toughness (kₘₐₓ) could be improved by using the binder, while the bending strength and bending modulus remained constant.

1. A curable composition for dental purposes, the composition comprising, respectively based on the total weight of the composition:
a) from 0.1 to 10% by weight of a binder based on a monomeric monofunctional (meth)acrylate, in which the ester group has a carbon chain of 3 or more carbon atoms;
b) from 1 to 15% by weight of one or more core-shell impact modifiers; and
c) from 1 to 90% by weight of a matrix material; and
d) from 0.01 to 3% by weight of at least one initiator.

2. The curable composition according to claim 1, characterized in that the ester group of the binder has a maximum of 16 carbon atoms.

3. The curable composition according to claim 1, characterized in that the content of monomeric methyl (meth)acrylate and/or monomeric ethyl (meth)acrylate is less than 0.5% by weight, based on the total weight of the composition.

4. The curable composition according to claim 1, characterized in that the impact modifier or modifiers is/are selected from the group of core-shell particles based on silicone/(meth)acrylate and based on (meth)acrylate/(meth)acrylate.

5. The curable composition according to claim 1, characterized in that the composition includes up to 70% by weight of at least one difunctional urethane (meth)acrylate, based on the total weight of the composition.

6. The curable composition according to claim 1, characterized in that the initiator is selected from the group consisting of thermal initiators and photoinitiators.

7. The curable composition according to claim 1, characterized in that the composition further contains up to 60% by weight of an inorganic filler, based on the total weight of the composition.

8. The curable composition according to claim 1, characterized in that the composition further contains up to 60% by weight of an organic filler, based on the total weight of the composition.

9. The curable composition according to claim 1, characterized in that the composition further contains up to 10% by weight of coloring substances, based on the total weight of the composition.

10. The curable composition according to claim 1, characterized in that said composition is printable.

11. (canceled)

12. A dental product prepared from a curable composition according to claim 1.

13. A process for producing a dental product by introducing a curable composition according to claim 1 into a device for additive manufacturing, and curing it.

14. The curable composition according to claim 7, wherein the inorganic filler is selected from the group consisting of silica, zirconia, aluminum oxide, alumina, apatite, titanium dioxide, and glass microbeads.

15. The curable composition according to claim 8, wherein the organic filler comprises (meth)acrylate beads.

16. The curable composition according to claim 15, wherein the (meth)acrylate beads comprise PMMA beads.

17. The curable composition according to claim 15, wherein the (meth)acrylate beads comprise rubber beads.

18. The curable composition according to claim 9, wherein the coloring substances comprise an organic substance.

19. The curable composition according to claim 9, wherein the coloring substances comprise an inorganic substance.
